(19) United States
(12) Patent Application Publication
(10) Pub. No.: US 2014/0288476 A1
(43) Pub. Date: Sep. 25, 2014

(54) CRANIAL CAP

(71) Applicants: Devin Bradford Wright, Portland, OR (US); Frances Patrick Delashaw, Orange, CA (US)

(72) Inventors: Devin Bradford Wright, Portland, OR (US); Frances Patrick Delashaw, Orange, CA (US)

(21) Appl. No.: 14/221,802

(22) Filed: Mar. 21, 2014

Related U.S. Application Data
(60) Provisional application No. 61/804,911, filed on Mar. 25, 2013.

(51) Int. Cl. A61F 13/00; A61F 13/12

(52) U.S. Cl. CPC A61F 13/00063; A61F 13/12; A61F 13/00068
USPC 602/48; 604/543

(57) ABSTRACT

Cranial caps designed to serve as an infection barrier and that can easily be placed on and removed from a patient's head are provided herein. Preferred cranial caps include an aperture that can be opened and closed that allows medical personal to easily inspect and/or treat the wound while the patient is wearing the cranial cap.

### CROSS-REFERENCE TO RELATED APPLICATIONS

This application claims priority to Provisional Application Ser. No. 61/804,911, filed Mar. 25, 2013 and entitled “Cranial Cap”, which is hereby expressly incorporated by reference in its entirety.

### FIELD OF THE INVENTION

The embodiments herein relate to a cranial cap worn on a patient's head, which assists in the prevention of microbial infections of a head wound, such as during post-operative care.

### BACKGROUND

Certain medical procedures performed on a patient's cranium, such as craniotomies, ear surgery, brain surgery, facelift surgery, or other similar methods, can leave wounds on the head. These wounds are susceptible to infection after the surgical procedure is completed, when the patient is recovering post-op. Additionally, medical caregivers often need to periodically inspect and treat these wounds during recovery to ensure that the patient is properly healing.

Current scalp care after a surgical procedure, such as a craniotomy, is not consistent among hospitals, because there are disadvantages in the current protocols. For example, some surgeons prefer applying an antibiotic ointment to the wound, without a dressing. This method is disadvantageous, in that it is not aesthetically pleasing to visiting friends and family members who view the visible head wound, and the patient may scratch or otherwise touch the ointment area, rub it against a pillow, etc., thereby diminishing the effectiveness of the ointment which can lead to contamination. Unfortunately, there are also disadvantages to using barriers on the head wound. For example, some surgeons wrap the head wound with a KERLIX® or KLING® turban. This is a time consuming procedure during the initial covering of the wound and when rewrapping the wound after periodic inspections during recovery. These types of turbans are also susceptible to blood leaking through and showing on the external surface of the wraps. As with the ointment method above, this is aesthetically undesirable. Furthermore, these types of dressings are best secured only when the patient's head is shaved, which is undesirable and often otherwise unnecessary. Still another way surgeons try to prevent post-surgical infection on head wounds is to apply an adhesive bandage over the wound. This method is disadvantageous in that it requires the patients head to be shaved, when it otherwise need not be. It also requires new adhesive bandages to be applied after inspection.

Accordingly, there is a need in the art to provide improved and quick head wound coverage without adhesives, which helps prevent microbial infection, allows for easy inspections by medical personal, helps prevent bleeding, and is aesthetically pleasing.

### SUMMARY OF THE INVENTION

Preferred embodiments are directed to a cranial cap, comprising a base section having an outer and inner layer of fabric and defining a lower rim on the cap sized to fit around the patient's head, an upper dome section having an outer and inner layer of fabric and sized to cover the upper sides and top of a patient's head, wherein the base section is coupled to and positioned below said upper dome section, an aperture that can be opened and closed and positioned either on the base or the upper dome section; and wherein the inner layer of fabric on the upper dome section is made of a synthetic polymer impregnated with silver and lacking elastomer fibers.

Additional embodiments are directed to methods of covering a head wound comprising performing a surgical procedure on a patient's head that results in a surgical wound on the patient's head; providing a cranial cap, comprising a base section having an outer and inner layer of fabric and defining a lower rim on the cap sized to fit around the patient's head, an upper dome section having an outer and inner layer of fabric and sized to cover the upper sides and top of a patient's head, wherein the base section is coupled to and positioned below said upper dome section, an aperture that can be opened and closed and positioned either on the base or the upper dome section; and wherein the inner layer of fabric on the upper dome section is made of a synthetic polymer impregnated with silver and lacking elastomer fibers; and placing the cranial cap over the patient's head after the surgical procedure is completed, whereby the wound on the patient's head is sufficiently covered by the cranial cap.

### BRIEF DESCRIPTION OF THE DRAWINGS

It will be appreciated that the drawings are not necessarily to scale, with emphasis instead being placed on illustrating the various aspects and features of embodiments of the invention, in which:

FIG. 1 is a perspective top view of a preferred cranial cap with the aperture closed.

FIG. 2 is a perspective top view of a preferred cranial cap with the aperture opened.

FIG. 3 is a perspective front view of a preferred cranial cap.

FIG. 4 is perspective view of the inside of a preferred cranial cap.

FIG. 5 shows a patient wearing the preferred cranial cap with the aperture open.

FIG. 6 shows a patient wearing the preferred cranial cap with the aperture closed.

### DETAILED DESCRIPTION OF THE ILLUSTRATED EMBODIMENTS

Embodiments of the present invention are described below with reference to the above-described Figures. It is, however, expressly noted that the present invention is not limited to the embodiments depicted in the Figures, but rather the intention is that modifications that are apparent to the person skilled in the art and equivalents thereof are also included.

Preferred embodiments are directed to soft, pliable cranial caps configured to be worn on a patient's head, having a releasably closable aperture, and that include a silver impregnated inner layer of fabric lacking elastomer fibers made of a synthetic polymer, such as nylon.

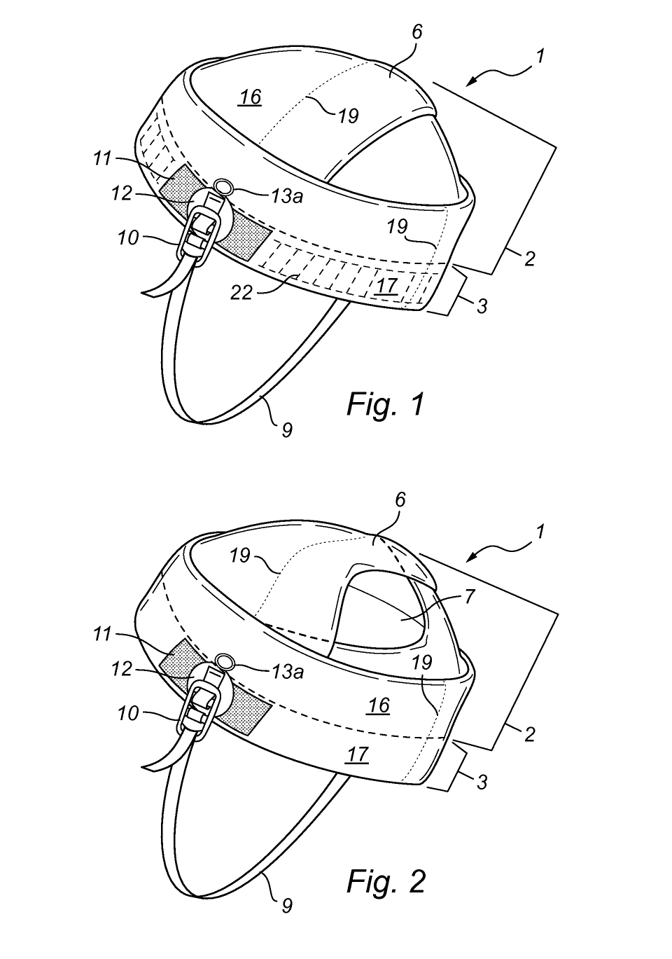

FIG. 1 shows a perspective view of a preferred cranial cap 1, including an upper dome shaped section 2 and a lower base section 3 surrounding a large opening 18 sized to fit over a patient's head. Both the upper dome 2 and lower base 3 are coupled together. According to preferred embodiments, and as shown in FIGS. 1-6, both the upper dome 2 and the base 3 include inner surfaces (8 and 15) and outer surfaces (16 and 17), each preferably made of pliable fabric. More specifically, it is preferred that the outer surface 16 of the upper dome 2 is made of a different material than the inner surface 8 of the upper dome 2. Likewise, it is preferred that the outer surface 17 of the base 3 is made of a different material than the inner surface 15 of the base 3.

Even more specifically, the inner surface 8 of the upper dome 2 is a fabric made of a synthetic polymer impregnated with silver and lacking elastomer fibers and adhesives. According to preferred embodiments, over 80% of the surface area of the inner surface 8 of the upper dome 2 is a fabric made of a synthetic polymer impregnated with silver and lacking elastomer fibers, but preferably over 90%, and even more preferably 100%, or substantially so. More specifically, it is preferred that the upper dome's inner surface 8 lacks any silver fabric having elastomer fibers. As are well known in the art, “elastomer fibers” are polymer fibers having individual elasticity; non-exclusive examples include: spandex (also known as elastane and LYRCRA®), elastic polyester, unsaturated and saturated rubbers, and thermoplastic elastomers. Advantageously, the fabric having embedded silver within it positioned on the inner surface 8 of the dome 2, is 100% nylon (without any elastomers), or 100% other synthetic polymer (without any elastomers). According to other embodiments, this fabric can be a nylon blended with cotton. According to non-preferred embodiments, the inner surface 8 of the upper dome 2 can alternatively contain elastomer fibers for all embodiments described herein.

Silver impregnated nylon has been shown to kill many types of bacteria on contact, including Staphylococcus Aureus and Methicillin Resistant Staphylococcus Aureus.

According to preferred embodiments, no more than a single inner layer of non-elastic silver fabric is used as the inner surface 8 in the dome section 2. According to further embodiments, the inner surface 8 of the dome section 2 can be a knit or woven fabric, but is preferably knit. A knit construction of non-elastic fibers allows for mechanical stretch and recovery of the fabric as a whole. Knit construction also allows for more cranial skin contact compared to a woven construction. According to preferred embodiments, the cap 1 is placed over a patients head such that the inner layer 8 of the upper dome 2 is directly contacting the head wound 20.

Preferably, the fabric of the outer surface 16 of the dome 2 and the inner surface 8 of the dome 2 are only single layers of fabric. Additionally, it is preferred that the outer surface 16 of the dome 2 and the inner surface 8 of the dome 2 are only coupled to together at the seams 19 defining the perimeter of fabric panels. The seams 19 are preferably stitched, but can additionally or alternatively be secured otherwise. Accordingly it is preferred that the majority of the surface area of the outer surface 16 and the inner surface 8 are not coupled together, such that the outer surface 16 and the inner surface 8 can be independently be bunched irrespective of each other.

Preferably, the outer layer 16 of the dome 2 is made of elastomer fibers, and lacks silver, such that it is has more stretch than the inner layer 8 of the dome section 2. According to preferred embodiments, as the majority of the surface area of the dome outer layer 16 is not coupled to the dome inner layer 8, the dome outer layer 16 can bunch, and stretch independent of the inner layer 8. The outer layer 16 can be made of many different materials, non-exclusively including polyurethane, polyester, and spandex and is preferably a blend of polyester and LYRCRA®. The outer layer 16 can be laminated such as with a polyurethane film. The outer layer 16 of the dome 2 serves multiple important functions. Firstly, it serves as an environmental protective barrier that protects the patient's wound from external contaminants. Additionally, it reduces movement of the inner layer 8 of the dome 2 so as to maintain improved contact between the silver fabric within the inner layer 8 and the wound 20. The outer layer 16 also preferably allows for the passage of air but not the passage of fluids also preferably in combination with the inner layer 8 to assist in preventing minor amounts of blood and other fluids from passing through the outer layer 16 of the dome section 2. According to non-preferred embodiments, the outer layer 16 of the upper dome 2 can alternatively lack elastomer fibers for all embodiments described herein.

The height of the base 3 section is preferably 0.5-3.5 inches or approximately so and includes an inner surface 15 and an outer surface 17 coupled to each other. The base 3 as a whole is preferably elasticized, to provide slight compression around the patient's head to help prevent the cap 1 from inadvertently falling off, or becoming misaligned. The slight elasticity of the base 3 allows care givers to easily place the cap 1 on the patient's head and to remove the cap 1 when needed. Preferably, the inner layer of the base 3 is only 1 layer and is made fabric made of a synthetic polymer impregnated with silver and lacking elastomer fibers and adhesives. According to preferred embodiments, over 80% of the surface area of the inner surface 15 of the base 3 is a fabric made of a synthetic polymer impregnated with silver and lacking elastomer fibers, but preferably over 90%, and even more preferably 100%, or substantially so. More specifically, it is preferred that the base's inner surface 15 lacks any silver fabric having elastomer fibers. As are well known in the art, “elastomer fibers” are polymer fibers having individual elasticity; non-exclusive examples include: spandex (also known as elastane and LYCRA®), elastic polyester, unsaturated and saturated rubbers, and thermoplastic elastomers. Advantageously, the fabric having embedded silver within it positioned on the inner surface 15 of the base 3, is 100% nylon (without any elastomers), or 100% other synthetic polymer (without any elastomers). According to other embodiments, this fabric can be a nylon blended with cotton. According to preferred embodiments, the inner surface 8 of the dome 2 and the inner surface 15 of the base 3 are the same material. According to non-preferred embodiments, the inner surface 15 of the base 3 can alternatively contain elastomer fibers for all embodiments described herein.

Preferably, the outer layer 17 of the base 3 is made of elastomer fibers, and lacks silver, such that it is has more stretch than the inner layer 15 of the base 3. The outer layer 17 can be made of many different materials, non-exclusively including polyurethane, polyester, and spandex, and preferably sueded poly LYRCA®. The outer layer 17 of the base 3 serves multiple important functions. Firstly, it serves as an environmental protective barrier that protects the patient's wound from external contaminants. Additionally, it reduces movement of the inner layer 15 of the base 3 so as to maintain improved contact between the silver fabric and the wound 20. The outer layer 17 also preferably allows for the passage of air but not the passage fluids in combination with the inner layer 3 to assist in preventing minor amounts of blood and other fluids from seeping out and passing through the outer layer 17 of the base section 3. According to non-preferred embodiments, the outer layer 17 of the base 3 can alternatively lack elastomer fibers for all embodiments described herein.

To conform fit around a patient's lower head, there can be an elastic fabric 22, such as a 1.5 inch wide fabric that wraps around the base, or over 80% of the base, inserted in between the inner layer 15 and outer layer 17 of the base 3.

Preferably, both of the inner surfaces 8 and 15 lack adhesives, non-exclusively including glues, such as acrylates, including methacrylates and epoxy diacrylates (also known as vinyl resins). The lack of adhesives on the inner surfaces 8 and 15 is advantageous as it allows for easy removal of the cap 1 from the patient's head, and does not require a shaved head for proper placement. According to preferred embodiments, the cranial caps 1 described herein, do not rely on tight compression to work, and thus lack inflatable means such as an inflatable bladder, or polystyrene (or other means to make the cap rigid).

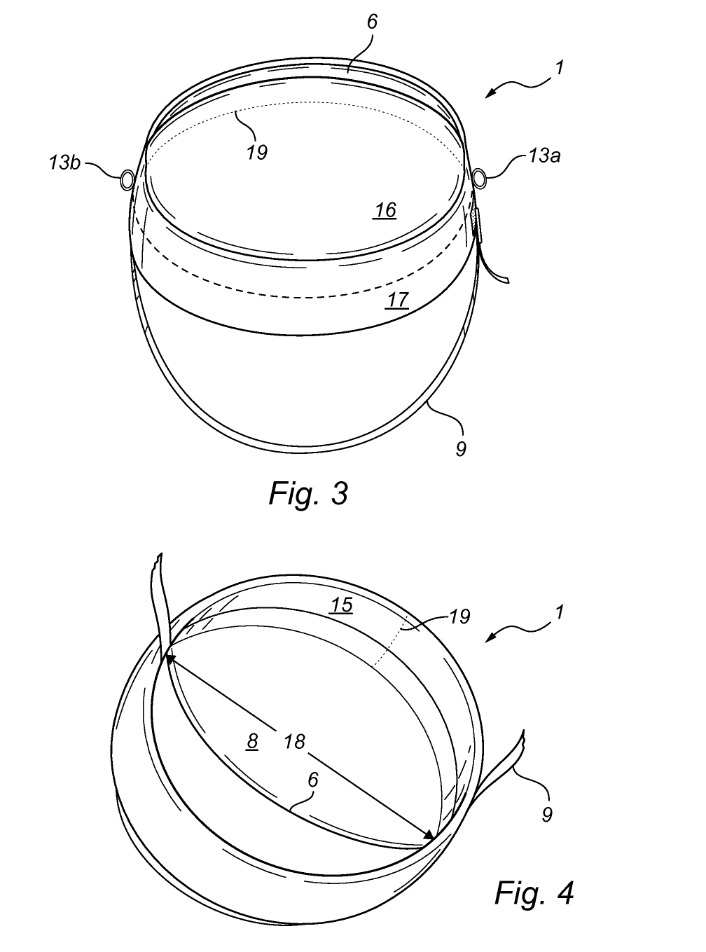

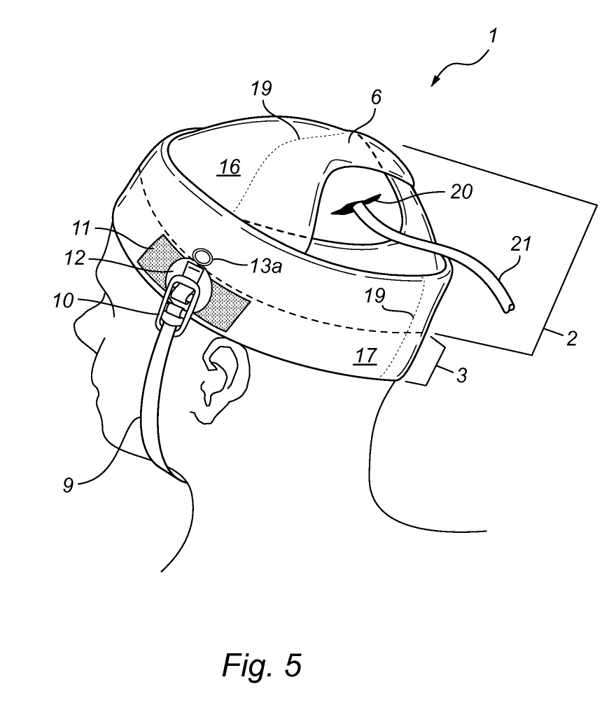

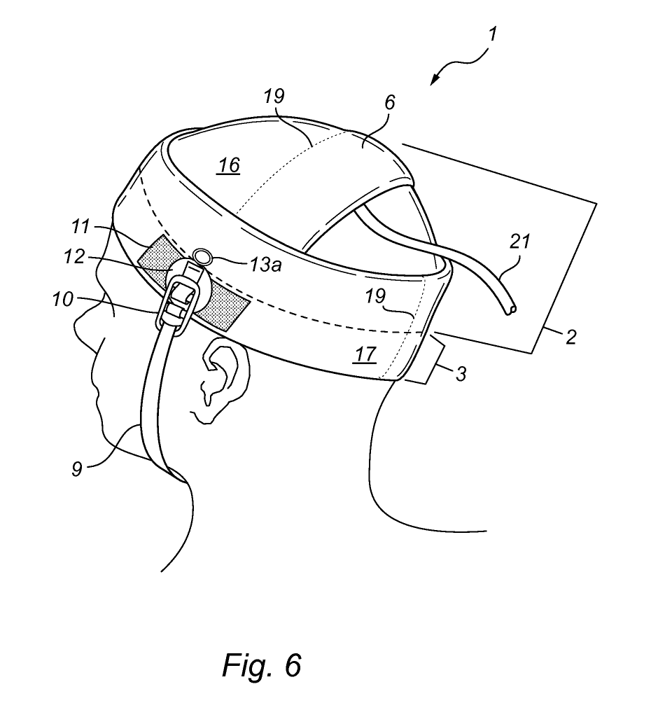

As shown in the FIGS. 1 and 2, an aperture 7 is positioned on the cranial cap 1. While preferably positioned on the dome section 2, and more preferably on top of the dome section 2 (as shown in FIGS. 1 and 2), the aperture 7 can be positioned at any suitable place along the cap 1. According to advantageous embodiments, the aperture 7 is positioned directly over the wound 20 on the patient's head. This positioning allows a medical provider to easily inspect, clean, drain, perform a ventriculostomy, or otherwise treat the wound 20 quickly during recovery. According to more specific embodiments, the aperture 7 is configured to be opened and closed quickly. One non-exclusive way to do this is to provide a flap 6 on the cap 1 that overlaps the aperture 7 and that can be pulled back without taking off the entire cap 1 from the patient's head. The flap 6 thus provides a barrier over the aperture 7, when the wound 20 is not being inspected and/or treated by a health care provider, but can be easily opened for these purposes. The flap 6 is also beneficial when a drainage tube 21 or other medical device is in contact with the wound for a prolonged period. (See FIGS. 5 and 6). More specifically, a pliable flap 6 allows the tube 21 to exit the cap 1 but still provide substantial coverage over the wound 20 during drainage. While a flap 6 by itself can be sufficient for releasably covering the aperture 7, it is additionally contemplated that additional and/or alternative means can be used to releasably open and close the aperture 7 with or without the flap 6, such as buttons, zippers, snaps, and hook and loop fasteners, such as VELCRO®. Preferably the aperture is about 7 to 8 inches in length, and can be opened and/or stretched to reveal a visible area of between 4 to 10 square inches. The flap 6 can be made of any suitable materials, but the inner surface of the flap 6 is preferably made of the same material as the inner surface 8 of the dome 2 and the outer surface of the flap 6 is preferably made of the same material as the outer surface 16 of the dome 2.

A chin strap 9 can be utilized that connects the left and right sides of the cap 1 and is of sufficient length to traverse below a patient's chin. Means for releasably coupling and/or tightening the chin strap 9 to one side of the cap 1 can also be used, such as snaps, buckles, ladders, and hook and loop fasteners such as VELCRO® and the like, for example. More specifically, the chin strap 9 can be coupled to a first fastening member 10 that releasably attaches to a complementary second fastening member 12 such that it can be fastened to the base 3 of the cap 1. The fastening members 10 and 12 can be any suitable releasable attachment means including hook and loop fasteners, snaps, etc. A ladder buckle 10 is also shown as one exemplary way of tightening the chin strap 9. The cap 1 can optionally include eyeglass holders 13a and 13b which can be small loops where the temples of the eyeglasses can be threaded through when the patient is not wearing them.

The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

What is claimed is:

1. A cranial cap, comprising:
(a) A base section having an outer and inner layer of fabric and defining a lower rim on the cap sized to fit around the patient's head,
(b) an upper dome section having an outer and inner layer of fabric and sized to cover the upper sides and top of a patient's head, wherein the base section is coupled to and positioned below said upper dome section,
(c) an aperture that can be opened and closed and positioned either on the base or the upper dome section; and
(d) wherein the inner layer of fabric on the upper dome section is made of a synthetic polymer impregnated with silver.

2. The cranial cap of claim 1, wherein the inner layer of fabric on the base section is made of a synthetic polymer impregnated with silver and lacking elastomer fibers.

3. The cranial cap of claim 2, wherein the inner layers of the base and upper dome lacks adhesives.

4. The cranial cap of claim 3, wherein the cap lacks means for inflation.

5. The cranial cap of claim 1, wherein the aperture is positioned on the dome section of the cap.

6. The cranial cap of claim 5, wherein the aperture comprises an overlapping hinged pliable flap that covers the aperture in a natural position but can be lifted such that a medical provider can view into the aperture.

7. The cranial cap of claim 6, wherein the inner layer of the flap is made of the same material as the inner layer of the dome, and the outer layer of the flap is made of the same material as the outer layer of the dome.

8. The cranial cap of claim 1, wherein the outer layer of the dome section and the inner layer of the dome section are only coupled along the perimeter of the fabric panels, such that the outer surface and the inner surface can be independently be bunched irrespective of each other.

9. A method of covering a head wound comprising:
(a) performing a surgical procedure on a patient's head that results in a surgical wound on the patient's head;
(b) providing a cranial cap, comprising
a base section having an outer and inner layer of fabric and defining a lower rim on the cap sized to fit around the patient's head,
an upper dome section having an outer and inner layer of fabric and sized to
cover the upper sides and top of a patient's head, wherein the base section is coupled to and positioned below said upper dome section,
an aperture that can be opened and closed and positioned either on the base or the upper dome section; and
wherein the inner layer of fabric on the upper dome section is made of a synthetic polymer impregnated with silver; and
(c) placing the cranial cap over the patient's head after the surgical procedure is completed, whereby the wound on the patient's head is sufficiently covered by the cranial cap.

10. The method of claim 9, wherein the cap is placed on the patient's head such that the aperture is aligned over the top of the wound, whereby a health care provider can open the aperture to inspect and treat the wound.

11. The method of claim 10, wherein the aperture comprises an overlapping hinged pliable flap that covers the aperture in a natural position but can be lifted such that a medical provider can view into the aperture.

12. The method of claim 11, further comprising placing a drainage tube into the wound to drain the wound, closing the flap to still cover while drainage tube is in contact with the wound.

13. The method of claim 11, wherein the inner layer of the flap is made of the same material as the inner layer of the dome, and the outer layer of the flap is made of the same material as the outer layer of the dome.

14. The method of claim 10, wherein the cranial cap's inner layer of fabric on the base section is made of a synthetic polymer impregnated with silver and lacking elastomer fibers.

15. The method of claim 10, wherein the inner layers of the base and upper dome lacks adhesives.

16. The method of claim 10, wherein the cranial cap lacks means for inflation.

17. The method of claim 10, wherein the inner layer of fabric on the base section of the cranial cap is made of a synthetic polymer impregnated with silver and lacking elastomer fibers.

18. The method of claim 10, wherein the outer layer of the dome section and the inner layer of the dome section are only coupled along the perimeter of the fabric panels, such that the outer surface and the inner surface can be independently be bunched irrespective of each other.

19. The cranial cap of claim 1, wherein the inner layer of fabric on the upper dome section lacks elastomer fibers.

20. The method of claim 9, wherein the inner layer of fabric on the upper dome section lacks elastomer fibers.
